(19) United States
(12) Patent Application Publication
(10) Pub. No.: US 2020/0261688 A1
(43) Pub. Date: Aug. 20, 2020

(54) METHOD FOR REDUCING STRESS, ANXIETY OR DEPRESSION, FOR BEHAVIOR MODIFICATION, AND FOR AIDING INFORMATION RETENTION

(71) Applicant: Christopher F. Thoma, Wildwood, MO (US)

(72) Inventor: Christopher F. Thoma, Wildwood, MO (US)

(21) Appl. No.: 16/278,468

(22) Filed: Feb. 18, 2019

(51) Int. Cl. A61M 21/02; G09B 5/04

(52) U.S. Cl. CPC A61M 21/02; A61M 2021/0027; G09B 5/04

(57) ABSTRACT

A method for reducing stress, anxiety and/or depression, for behavior modification and for information retention and/or recall in a subject is provided herein. The method comprises receiving a mono auditory stimulus in an ear of the subject and the subject performing cross-body stimulation, such as cross-body tapping.

### FIELD

The present disclosure relates to a method for reducing stress, anxiety, and/or depression, for behavior modification and for aiding information retention.

### STATEMENT REGARDING PRIOR DISCLOSURE BY THE INVENTOR

The inventor has disclosed aspects of a method described herein on 10 May 2018 on www.neuroredeem.com.

### BACKGROUND

Anxiety disorders are the most common mental illness in the United States, affecting 40 million adults in the United States age 18 and older, or 18.1% of the population every year. People with an anxiety disorder are three to five times more likely to go to the doctor and six times more likely to be hospitalized for psychiatric disorders than those who do not suffer from anxiety disorders. Anxiety disorders develop from a complex set of risk factors, including genetics, brain chemistry, personality, and life events.

It is not uncommon for someone with an anxiety disorder to also suffer from depression or vice versa. Nearly one-half of those diagnosed with depression are also diagnosed with an anxiety disorder. Depression is the leading cause of disability worldwide. Almost 75% of people with mental disorders remain untreated in developing countries with almost 1 million people taking their lives each year. In addition, according to the World Health Organization (WHO), 1 in 13 globally suffers from anxiety. The WHO reports that anxiety disorders are the most common mental disorders worldwide with specific phobia, major depressive disorder and social phobia being the most common anxiety disorders.

Both natural and pharmacological-based products and methods have been developed for reducing stress, anxiety and/or depression. Examples of existing methods for reducing stress, anxiety and/or depression include meditation, exercise, therapy, hypnosis, acupuncture and listening to music. Additionally various natural dietary supplements and pharmacological medication exist to reduce stress, anxiety and/or depression. However, a need exists for alternative and improved methods to reduce stress, anxiety and/or depression, for example methods that help overcome the lack of focus issues found with traditional methods for reducing stress, anxiety and/or depression.

### SUMMARY

This section provides a general summary of the disclosure, and is not a comprehensive disclosure of its full scope or all of its features.

In various aspects, a method of reducing stress, anxiety and/or depression in a subject in need thereof is disclosed herein. The method comprises receiving a mono auditory stimulus in a first ear of the subject; and the subject performing cross-body stimulation.

In particular embodiments, the cross-body stimulation may comprise tapping a body part on the opposite side of the first ear receiving the mono auditory stimulus with a body part on the same side as the first ear receiving the mono auditory stimulus. This is also known as cross-body tapping.

In particular embodiments, the method can be used in a subject having PTSD, ADD, ADHD or autism.

Additionally, the method may be used for behavior modification. In particular embodiments, the method can be used for reducing addictive behavior, modifying diet and/or exercise, or enhancing athletic performance.

Additionally, the method may be used to help a subject, for example a student, maintain or retain information.

Further areas of applicability will become apparent from the description provided herein. The description and specific examples in this summary are intended for purposes of illustration only and are not intended to limit the scope of the present disclosure.

### DETAILED DESCRIPTION

### I. Definitions

The term “and/or” as used in a phrase such as “A and/or B” herein is intended to include “A and B”, “A or B”, “A”, and “B”.

The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

It should be understood for any recitation of a method, composition, device, or system that “comprises” certain steps, ingredients, or features, that in certain alternative variations, it is also contemplated that such a method, composition, device, or system may also “consist essentially of” the enumerated steps, ingredients, or features, so that any other steps, ingredients, or features that would materially alter the basic and novel characteristics of the invention are excluded therefrom.

Although the terms first, second, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

The term “cross hemisphere activation” or “cross hemisphere stimulation” of the brain as used herein refers to when a stimulus enters a neurological pathway on one side or hemisphere of the brain and crosses a commissure (nerve bundle) to the other side or hemisphere of the brain. For example, information from a stimulus may enter the body on one side of the body and is transmitted across the brainstem or corpus callosum to the other side of the brain where it is then processed. Neurological information can then be disseminated throughout associated neurological tissues. The more information that crosses a commissure of the brain the more neurological activation takes place which can be demonstrated by fMRI. Cross hemisphere activation or stimulation can also be synonymous with bi-lateralization of brain function, bilateral brain connectivity, bilateral brain activation/stimulation, bilateral neurological conduction, gross cognitive activation/stimulation, cross pons activation/stimulation, cross vermis activation/stimulation, cross corpus callosum activation/stimulation and cross commissure activation/stimulation.

As used herein, “induction” refers to the process used to create a mono-cognitive state, a hypnotic state or a trance state in a subject.

As used herein, “confusion induction” refers to the process of creating a mono-cognitive state, a hypnotic state or a trance state by utilizing cognitively disruptive, aberrant single or multiple simultaneous cognitive inputs so that the conscious mind disassociates from the subconscious allowing for heightened focus, concentration, and suggestibility.

As used herein, “sensory overload induction” refers to a process in which the conscious mind is given more information than it can handle at one time and the conscious mind disassociates from the subconscious allowing for heightened focus, concentration, and suggestibility.

As used herein, “hypnosis” refers to an induced state of consciousness in which the subject's subconscious and conscious mind have greater disassociation allowing for heightened focus, concentration, and suggestibility.

As used herein, “mono-cognition” or “mono-cognitive state” refers to a trance like state that occurs when the nervous system becomes overwhelmed. The subconscious looks for a single (mono) solution to this new situation. Mono-cognition occurs after a sensory overload induction and is a hyper focused state that acts like a reboot of the subconscious. It typically lasts for less than 30 sec. As used herein, mono-cognition is not the same as the hypnotic state referred to as “mono-idealism” by a hypnotherapist.

As used herein, “mono auditory or unilateral stimulation” refers to a verbal message and/or sound received in only one ear at a time, as opposed to both ears at the same time.

As used herein, “cross-body stimulation” refers to a body part on one side of a subject's body indirectly or directly touching a body part on the opposite side of the subject's body, such as the left hand touching the right leg, or vice versa. This can be referred to as cross-body tapping. Cross-body stimulation can also refer to a subject's movement and/or touch that occurs on the opposite side of the body as an auditory stimulus. For example, when an auditory stimulus is received in the left ear and a subject move's a body part such as their eyes or neck to the right side, and vice versa.

As used herein, “tapping” refers to touching an area of the body for a period of time, usually a short 1-2 second period of time. It may be performed following receiving a command to touch an area of the body. Tapping can be very complex or can be as simple as using a repetitive finger motion against another body part.

### II. Method

The method described herein may stimulate the brain through cross hemisphere activation, using mono auditory stimulation and tapping, as a confusion induction and/or sensory overload induction, to create a mono-cognitive state, a hypnotic state or a trance state to help teach individuals to calm their minds, increase focus, and/or learn new behavioral patterns. Without being bound by theory, the greater the cross hemisphere stimulation, the stronger the induction of a mono-cognitive state, hypnotic state or trance state. These states, particularly mono-cognition, may allow for a reset of the neurological system to a point of somnolence. Thus in some embodiments, the method described herein may induce a mono-cognitive state, a hypnotic state or a trance state in a subject. In a particular embodiment, the method described herein may induce a mono-cognitive state.

In some embodiments, the method can induce a stronger more powerful hypnotic trance state more efficiently than those found in traditional stereo auditory hypnotic inductions. Without being bound by theory, this efficiency may be achieved by improving confusion induction in the subject.

### Mono Auditory Stimulus

The method may comprise receiving a mono auditory stimulus in an ear of a subject. The mono auditory stimulus is received in one ear only (left or right) at a time. That is to say, the mono auditory stimulus is not received in both ears at the same time, but can be received in the same ear consecutively (e.g. left, left, left), or alternate between ears (e.g. left, right, left). Without being bound by theory, a mono auditory stimulus may aid in processing of the stimulus across the corpus callosum and allow for the use of an induction process to create a state of mono-cognition or extreme focus.

In some embodiments, the mono auditory stimulus may comprise a command to perform cross-body stimulation, such as tapping. For example, the mono auditory stimulus may comprise a verbal command to tap one body part using another body part. In a particular embodiment, the mono auditory stimulus may comprise a command to perform cross-body tapping. Additionally or alternatively, the mono auditory stimulus may comprise a tone or sound that has previously been associated or anchored with an action that increases neurological stimulation across the corpus callosum. For example, in some embodiments, the mono auditory stimulus may be a tonal command associated with turning the subject's head/face/neck in one direction while the eyes are focused in the opposite direction of the turning of the head/face/neck. In particular embodiments, the subject can receive a mono auditory stimulus in one ear, such as tonal command or verbal command, and then the subject can move their head/face/neck to the opposite side of the body of the ear receiving the mono auditory stimulus and move their eyes to the same side of the body as the ear receiving the mono auditory stimulus.

In some embodiments, the mono auditory stimulus may be a verbal recording, for example, a recording on any suitable device or media capable of emitting an audible signal such as an e-book, audio session, video, podcast/netcast, compact disc, etc. . . . . Additionally or alternatively, the mono auditory stimulus may be spoken to the subject in a “live” setting, for example in an educational or therapy setting.

### Cross-Body Stimulation

Following receiving the mono auditory stimulus, such as a verbal or tonal command to perform cross-body stimulation, the subject may then perform the cross-body stimulation. That is to say, in some embodiments, the cross-body stimulation can substantially occur after receiving the mono auditory stimulus. In these instances, the subject follows the direction of the mono auditory stimulus, e.g. the command to perform cross-body stimulation.

In some embodiments, cross-body stimulation may comprise cross-body tapping such that the subject may tap a body part on the opposite side of the ear receiving the mono auditory stimulus with a body part on the same side as the ear receiving the mono auditory stimulus. For example, the subject may receive a command or tone in the right ear to use their right hand to tap their left leg or vice versa, and then performs the tapping according to the command or tone.

The body part being tapped may be any body part such as a leg, foot, toe, arm, hand, finger, torso, neck, face, ear or scalp. Additionally, the body part doing the tapping may be any body part including a hand, finger, foot or toe.

The tapping may be performed directly or indirectly with a body part. In some embodiments, the tapping is performed directly as in the example above, when a right hand will tap a left leg. Additionally or alternatively, the tapping may be performed indirectly. A hand/finger(s) or toe(s) can grip a device/object, in which case the device/object physically taps the body part on the opposite side of the body. For example, a subject's right hand can be gripping a stick or pen and then tap the left leg with the stick or pen.

Additionally or alternatively, cross-body stimulation may comprise the subject moving the neck and/or eyes to the opposite side of the body of the ear receiving the mono auditory stimulus. For example, the subject may receive a command or tone in the right ear to move their eyes or neck to the left side of the body or vice versa, and then performs the movement according to the command or tone. This embodiment can be particularly useful for a subject who is paralyzed.

In some embodiments, the mono auditory stimulus and/or the cross-body stimulation can be repeated several times with the mono auditory stimulus being received in the same ear multiple times, i.e. repetitive cross-body stimulation with two or more commands or tones being received in either the right ear or the left ear. For example, as discussed above, the subject may receive a command or tone in the right ear to use their right hand to tap their left leg, and then performs the tapping according to the command or tone. Subsequently, the subject may receive another command or tone in the right ear to use a body part on the right side to tap a body part on the left side, and so forth. Additionally or alternatively in some embodiments, the subject may receive a command or tone in the right ear to turn their neck or move their eyes to the left side of the body, and then the subject performs the movement according to the command or tone. Subsequently, the subject may receive another command or tone in the right ear to turn their neck or move their eyes to the left side of the body. This “same ear” pattern may be repeated several times.

Additionally or alternatively, the mono auditory stimulus and/or cross-body stimulation may be repeated in an alternating pattern, i.e. repetitive cross-body stimulation with commands or tones alternating being received in the right ear and then the left ear or vice versa. For example, the subject may receive a command or tone in the right ear to use their right hand to tap their left leg and follows the command or tone. Subsequently, the subject receives a command or tone in the left ear to use their left hand to tap their right leg and follows the command or tone. Additionally or alternatively in some embodiments, the subject may receive a command or tone in the right ear to turn their neck or move their eyes to the left side of the body, and then the subject performs the movement according to the command or tone. Subsequently, the subject may receive another command or tone in the left ear to turn their neck or move their eyes to the right side of the body. This “alternating” ear pattern may be repeated several times.

The second or subsequent mono auditory stimulus may be the same or different as the previous mono auditory stimulus, so long as cross-body stimulation occurs. It is also possible to mix same ear and alternating ear mono auditory stimulus patterns.

The subject may receive the mono auditory stimulus and/or perform cross-body stimulation once or more. For example, the mono auditory stimulus and/or cross-body stimulation may be repeated at least two, at least three, at least four, at least five, at least six, at least seven, at least eight, at least nine or at least 10 or more times; or for example, 1 to 10 times.

### Uses

The method provided herein has multiple uses. The method may be used to help reduce the symptoms of mental disorders in a subject in need thereof, such as neurodevelopmental disorders, psychotic disorders, mood disorders, anxiety disorders and combinations thereof. In a particular embodiment, the method may reduce stress, anxiety and/or depression in a subject having one or more of these mental disorders.

In some embodiments, the subject may have a neurodevelopmental disorder such as autism, attention deficit hyperactivity disorder (ADHD) and/or attention deficit disorder (ADD), dyslexia and/or Tourette's.

In some embodiments, the subject may have a psychotic disorder such as schizophrenia; or a mood disorder such as bipolar disorder and clinical depression. In a particular embodiment, the method can be used to reduce depression in a subject in need thereof.

In some embodiments, the subject may have an anxiety disorder. The anxiety disorder can be any anxiety disorder found in the DSM-5 list of anxiety disorders. DSM-5 is the published list of mental disorders by the American Psychiatric Association's Diagnostic and Statistical Manual of Mental Disorders. For example, the anxiety disorder may be a phobia, a panic disorder, agorapobia, post-traumatic stress disorder (PTSD), generalized anxiety disorder, anxiety disorder due to another medical condition, substance/medication-induced anxiety disorder, selective mutism, separation anxiety disorder, other specified anxiety disorder or unspecified anxiety disorder. In particular embodiment, the method can reduce stress and anxiety in a subject having PTSD. In another particular embodiment, the method can reduce or eliminate a phobia in a subject.

In some embodiments, the subject may be hypnosis-resistant.

In some embodiments, the subject may be paralyzed.

Additionally or alternatively, the method provided herein may be used for behavior modification in a subject, i.e. to change a negative behavior pattern. For example, the method may be used to reduce any addictive behavior such as an addiction to smoking, drugs, alcohol and/or gambling.

Additionally or alternatively, the method provided herein may be used to modify diet and exercise in a subject. For example, the method can be used in a subject desiring to have or maintain a more healthy lifestyle by eating a healthier diet and/or exercising.

Additionally or alternatively, the method provided herein may be used for aiding information retention in a subject in need thereof. The method can be used to maintain and/or enhance information retention and/or recall in a subject in need thereof. The subject may have a need to maintain or enhance information retention and/or recall for performance or testing. For example, a student may use a method provided herein in an educational setting to help retain and/or recall information. Additionally or alternatively, the subject may have dementia such as early stage dementia and the method can be used to help maintain and/or enhance information retention and/or recall. The subject may receive a mono auditory stimulus, perform cross-body stimulation and then be given an associated action or recall mechanism to aid in information recall.

### Additional Steps

In some embodiments, the method may further comprise the subject receiving an educational message specific to the subject's disorder. For example, PTSD subjects may be given specific education on how to seek help when they feel overwhelmed. ADD and ADHD subjects may be given specific education on when it is appropriate to be active and when to be still. Autism subjects may be given specific education on how to properly react in a social setting in which they would normally react inappropriately. Anxiety subjects may be given specific education on how to notice stressful situations and to avoid them. Depression subjects may be given specific education on how to seek help from others when needed. These educational messages may include any social, physiological, academic, and behavioral information that is specific for any number of disorders.

Additionally or alternatively, the method may further comprise the subject receiving a behavior modification message specific to the behavior that is desired to be modified. For example, PTSD subjects may be given a specific behavior modification message on how to properly react in situations that previously triggered aberrant responses. ADD and ADHD subjects may be given a specific behavior modification message on how to properly react in situations that need greater focus than they are usually able to obtain. Autism subjects may be given a specific behavior modification message on how to look at individuals when speaking. Anxiety subjects may be given a specific behavior modification message to help with proper responses to stressful situations. Depression subjects may be given a specific behavior modification message to get physical activity when depressed. These behavior modification messages may contain any social, physiological, and behavioral information that is specific for any number of disorders.

The educational message or behavior modification message may be received after the mono auditory stimulus and cross-body stimulation has been performed once, or after the mono auditory stimulus and cross-body stimulation has been performed several times. The educational message or behavior modification message may be recorded in any suitable medium or delivered in a live setting, such as an educational and/or therapy setting.

Additionally or alternatively, the method may further comprise receiving guided meditation before receiving the mono auditory stimulus in the ear of the subject. Guided meditation may help with induction of the subject.

### EXAMPLES

The NeuroRedeem™ program can be used for various behavior modification modalities. The program consists of four audio sessions. The first audio session is an introduction to explain to the subject how to use the method of mono auditory stimulus and tapping. The second session teaches the subject how to reach the focused state of consciousness through guided meditation and begins repeating the mono auditory stimulus and tapping alternating between left and right ear to create a mono-cognitive state. The third session teaches the subject how to re-create the proper behavior without reaching the full mono-cognitive state. In the fourth session, the subject learns how to control and increase positive feelings without tapping and even with their eyes open. Each successive session reduces the need to be in a mono-cognitive state. As the subject goes through the sessions, they find it easier and easier to perform the proper behaviors even with multiple distractions. Once the new behavioral programs are taught to the point of being automatic, the subject can perform them with minimal effort or even without having to think about it.

The construct of the NeuroRedeem™ program remains the same for each use whereby the subject is equipped with headphones which are checked to confirm they are on correctly so that the language specific to the left or right side is heard in the left or right ear, respectively. The subject is given test commands to ensure they are able to follow commands. The subject is then taken through an individual process with mono auditory stimulus with commands to induce a hypnotic state. This induction process continues through deepening with further commands.

Once the induction is deepened, the subject is given behavior modification instructions according to their specific need. Depending on the situation, the subject maybe be given cross hemisphere stimulation again to solidify hypnotic suggestions. The subject is then awakened from the trance state and the process is continued in subsequent sessions.

### Example 1 (Anxiety)

For subjects with anxiety, the NeuroRedeem™ process identified above is followed with specific behavior modification instructions to reduce the number of inputs into a particular decision by suggesting affirmation of the statement “I focus on only those issues relative to this decision that I have control of.”

### Example 2 (PTSD)

For subjects with PTSD, the NeuroRedeem™ process identified above is followed with specific behavior modification instructions to change the reaction to a trigger in a public situation to an appropriate reaction.

### Example 3 (Information Retention/Recall)

For students wanting to enhance learning, the NeuroRedeem™ process identified above is followed with specific instructions to easily recall subconscious information accurately.

### Example 4 (Autism)

For subjects with autism, the NeuroRedeem™ process identified above is followed with commands to appropriately respond to specific expressions of individuals with whom they are carrying on a conversation.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of reducing stress, anxiety and/or depression in a subject in need thereof, the method comprising
receiving a mono auditory stimulus in a first ear of the subject; and
the subject performing cross-body stimulation.

2. The method of claim 1, wherein the cross-body stimulation comprises cross-body tapping or moving the neck of the subject to the opposite side of the body of the first ear receiving the mono auditory stimulus and the eyes of the subject to the same side of the body of the first ear receiving the mono auditory stimulus.

3. The method of claim 1, wherein the subject has a neurodevelopmental disorder, a mood disorder and/or an anxiety disorder.

4. The method of claim 3, further comprising the subject receiving an educational message specific to the neurodevelopmental disorder, the mood disorder and/or the anxiety disorder.

5. The method of claim 3, wherein the subject has autism, PTSD, ADD, ADHD and/or depression.

6. The method of claim 1, wherein the mono auditory stimulus comprises a command to perform the cross-body stimulation.

7. The method of claim 6, wherein the cross-body stimulation substantially occurs after receiving the command to perform the cross-body stimulation.

8. The method of claim 1, wherein a mono cognitive state, a hypnotic state or a trance state is induced in the subject.

9. The method of claim 1, further comprising repeating the receiving and the performing cross-body stimulation steps.

10. The method of claim 1, further comprising
receiving a mono auditory stimulus in a second ear of the subject; and
the subject performing cross-body stimulation.

11. The method of claim 10, wherein the mono auditory stimulus alternates between the first ear and the second ear.

12. The method of claim 11, further comprising repeating the receiving and the performing cross-body stimulation steps.

13. The method of claim 1, further comprising receiving guided meditation before receiving the mono auditory stimulus in the first ear of the subject.

14. A method for modifying behavior in a subject in need thereof, the method comprising
receiving a mono auditory stimulus in a first ear of the subject; and
the subject performing cross-body stimulation.

15. The method of claim 14, wherein the cross-body stimulation comprises cross-body tapping or moving the neck of the subject to the opposite side of the body of the first ear receiving the mono auditory stimulus and the eyes of the subject to the same side of the body of the first ear receiving the mono auditory stimulus.

16. The method of claim 14, wherein the subject has an addiction and the method reduces the addiction.

17. The method of claim 14, wherein the subject has a neurodevelopmental disorder, a mood disorder and/or an anxiety disorder.

18. The method of claim 14, wherein the behavior modification is a change in diet or exercise of the subject.

19. The method of claim 14, wherein the behavior modification is modifying athletic performance.

20. The method of claim 14, further comprising the subject receiving a behavioral modification message.

21. A method for aiding information retention in a subject in need thereof, the method comprising
receiving a mono auditory stimulus in a first ear of the subject; and
the subject performing cross-body stimulation.

22. The method of claim 21, wherein the cross-body stimulation comprises cross-body tapping or moving the neck of the subject to the opposite side of the body of the first ear receiving the mono auditory stimulus and the eyes of the subject to the same side of the body of the first ear receiving the mono auditory stimulus.

23. The method of claim 21, wherein the subject has a need to retain information for performance, testing, early stage dementia, and the subject has a need to recall information.

24. The method of claim 21, wherein the subject is given an associated action or recall mechanism to aid in information recall.
